(19) United States
(12) Patent Application Publication
(10) Pub. No.: US 2007/0116664 A1
(43) Pub. Date: May 24, 2007

(54) Methods and compositions for treating hair and skin afflictions

(76) Inventor: Shmuel Gonen, Kiryat Ono (IL)

Correspondence Address: PEARL COHEN ZEDEK LATZER, LLP, 1500 BROADWAY 12TH FLOOR, NEW YORK, NY, 10036, US

(21) Appl. No.: 11/601,788

(22) Filed: Nov. 20, 2006

Related U.S. Application Data
(60) Provisional application No. 60/738,023, filed on Nov. 21, 2005.

(51) Int. Cl. A61K 8/97; A61K 36/48; A61K 31/56

(52) U.S. Cl. 424/74; 424/757; 514/169

(57) ABSTRACT

Method and composition for promoting hair growth, preventing or minimizing hair loss and treating other hair and skin afflictions are disclosed. The methods include topical application of a composition comprising at least a phyto-steroid to the skin or hair follicles being treated.

### CROSS REFERENCE TO RELATED APPLICATIONS

This application claims benefit of U.S. Provisional Application No. 60/738,023 filed on Nov. 21, 2005, entitled, “METHODS AND COMPOSITIONS FOR TREATING HAIR AND SKIN AFFLICTIONS”, which is hereby incorporated by reference.

### FIELD OF THE INVENTION

The invention relates to methods and products for treating hair and skin conditions. In addition to treating hair condition, the invention comprises methods for promoting of hair growth, preventing, stopping or minimizing hair loss, conditioning the hair and scalp, thickening the hair, treating dandruff, etc. and skin conditions including smoothing of the skin, seborrhea dermatitis, psoriasis and other skin conditions, possibly including healing of wounds in the skin.

### BACKGROUND OF THE INVENTION

Dermatologists recognize many different types of hair loss, the most common by far being “alopecia” wherein human males begin losing scalp hair at the temples and on the crown of the head as they get older. While this type of hair loss is largely confined to males, hence its common name “male pattern baldness”, it is also known in women. Be that as it may, no known cure has yet been found despite continuing attempts to discover one.

Another factor that contributes to the end result is a change in the cycle of hair growth. All hair, both human and animal, passes through a life cycle that includes three phases, namely, (1) the anagen phase (2) the catagen phase and (3) the teiogen phase. The anagen phase is the period of active hair growth and, insofar as scalp hair is concerned, this generally lasts from 3-5 years. -The catagen phase -is a short transitional phase between the anagen and telogen phases which, in the case of scalp hair, lasts only 1-2 weeks. The final phase is the telogen phase which, for all practical purposes, can be denominated a “resting phase” where all growth ceases and the hair eventually is shed preparatory to the follicle commencing to grow a new one. Scalp hair in the telogen phase is also relatively short-lived, some 34 months elapsing before the hair is shed and a new one beings to grow.

Under normal hair growth conditions on the scalp, approximately 88% of the hairs are in the anagen phase, only 1% in catagen and the remainder in telogen. With the onset of male pattern baldness, a successively greater proportion of the hairs are in the telogen phase with correspondingly fewer in the active growth anagen phase.

While a good deal is known about the results of male pattern baldness, very little is known about its cause. About all that can be said is that the cause is felt to be genetic and hormonal in origin although, as will be seen presently, the known prior art attempts to control it through hormone adjustment have been singularly unsuccessful.

Another type of baldness is Alopecia areata which is a dermatogical condition of unknown etiology. The disease is manifested as circumscribed, non-inflamed areas of baldness on the scalp, eyebrows and bearded areas of the face. Severe forms of the disease are called alopecia totalis or alopecia universalis.

In the past, treatment of alopecia areata has involved the injection of corticosteriods, namely triamcinolone acetonide suspension, into the patches of dermatologic involvement. For more progressive disease states, the resort to systemic corticosteroid therapy has been made. For the most part, however, the above listed treatments are not warranted because of the serious side effects of corticosteroids as well as from a cost stand point unless there is serious emotional or economic impact on the patient as a result of the disease.

The remaining result associated with alopecia is the severe dimunition of hair follicles. A bald human subject will have in average only about 306 follicles per square centimeter, whereas, a non-bald one in the same age group (30-90 years) will still have an average of 460 follicles per square centimeter. This amounts to a one-third reduction in hair follicles which, when added to the increased proportion of vellus hair follicles and the increased number of hair follices in telogen, is both significant and noticeable. It is written that approximately 50% of the hairs must be shed to produce visible thinning of scalp hair. It is thus a combination of these factors: (1) transition of hairs from terminal to vellus, (2) increased number of telogen hairs-some of which have been shed, and (3) loss of hair follicles (atrophy in Settel's description) that produces “baldness”.

At the present time, one known treatment for male pattern alopecia is hair transplantation. Plugs of skin containing hair are transplanted from areas of the scalp where hair is growing to bald areas with reasonable success; however, the procedure is a costly one in addition to being time-consuming and quite painful. Furthermore, the solution is inadequate from the standpoint that it becomes a practical, if not an economic, impossibility to replace but a tiny fraction of the hair present in a normal healthy head of hair.

The androgenic hormone, testosterone, was known, for example, to stimulate hair growth when applied topically to the deltoid area as well as when injected into the beard and pubic regions. Even oral administration was found to result in an increased hair growth in the beard and pubic areas as well as upon the trunk and extremities. While topical application to the arm causes increased hair growth, it is ineffective on the scalp and some thinning may even result. Heavy doses of testosterone have even been known to cause male pattern alopecia (in male and female).

A wide variety of materials or compositions have been disclosed in the prior art as being effective for promoting hair growth and/or for preventing, stopping or minimizing hair loss as well as treating related conditions such as dandruff, itching of the scalp, etc.

A number of such treatment agents were disclosed in U.S. Pat. No. 4,874,791 issued Oct. 17, 1989 to Adachi, et al. In addition, that patent disclosed and claimed a hair-growing agent containing as an effective ingredient an aliphatic carboxylic acid having an odd number of carbon atoms or a derivative thereof.

U.S. Pat. No. 4,814,351 issued Mar. 21, 1989 to Mathews, et al. disclosed yet another scalp treatment for reducing average daily hair loss by periodically applying to the scalp a composition containing an active chelating agent. That reference was further of note in that its background statement set forth various theories for promoting hair growth.

U.S. Pat. No. 4,769,231 issued Sep. 6, 1988 to Ogura, et al. disclosed a hair tonic composition based upon the discovery that external or topical application of an extract from dong chong xia cao is capable of promoting hair growth.

U.S. Pat. No. 4,853,216 discussed the topical application of alpha1 adrenergic agonist for producing a pilomotor effect to enhance shaving.

Still other references have dealt with various active agents for treating related conditions including various skin conditions. For example, U.S. Pat. No. 4,719,226 issued Jan. 12, 1988 to Otsuka, et al. disclosed a percutaneous absorption type preparation and process for preparing the product. U.S. Pat. No. 4,725,609 issued Feb. 16, 1988 to Kull, Jr., et al. disclosed the topical use of nicotinamide to promote angiogenesis, reepithelialization and wound healing. U.S. Pat. No. 4,656,192 referred to ester derivatives of tropolone as being useful antimicrobial agents, hair growth stimulants and dental medicines.

U.S. Pat. No. 6,461,596 issued Oct. 8, 2002 to Taylor A., discloses a composition suitable for topical application. The composition is composed of an effective amount of spironolactone and an inert oleaginous carrier, and applied topically to the area of the body that increased hair growth is desired.

U.S. Pat. No. 6,380,168 issued Apr. 30, 2002 to Nomura Co. Ltd., discloses a composition which is useful for promoting hair growth. The hair growth promoter composition comprises derivative of 1H-indole, 1-benzofuran or 1-benzothiophene. Preferably, the compound comprises at least two saccharide moieties.

U.S. Pat. No. 6,203,782 issued Mar. 20, 2001 to Universal Biologics Inc., discloses a methods and compositions for promoting hair growth, including topical application of the compositions to the skin or hair follicles being treated. The compositions include as an essential component a compound selected from anole, anethole, analogs thereof, polymers thereof and mixtures thereof, may be found in herb families including umbelliferae, magnoliaceae, labiatae and rutaceae. The invention preferably contemplates selecting the treatment agent from the class of herbs consisting of Foeniculum vulgares (fennel seed), Pimpinella anisum (anise), Carum carvi (caraway seeds) and mixtures thereof with each other and/or other herbs.

In addition to the above references, various herbs have long been known in Chinese herbology or medicine as having various medicinal or physiological applications. In particular, various herbs have been discussed as being effective for controlling the quality and color of hair as well as being effective hair growth agents.

The following herbs have also been considered suitable for treating baldness in local or topical applications:

1. Gu sui bu-Rhizoma gusuibu (drynaria); and

2. Ce bai ye-Cacumen biotae orientalis.

In any event, continuing efforts to discover or develop such materials demonstrates a continuing need for compositions or materials which are effective for promoting hair growth, preventing or minimizing hair loss and for treating various skin conditions. It is of course particularly important that such materials or compositions be uniformly effective and safe to use in order to enhance their effectiveness for use by large numbers of people.

### SUMMARY OF THE INVENTION

In an embodiment of the invention a method for treating hair and skin conditions including promoting hair growth, preventing, stopping or minimizing hair loss, conditioning the hair and scalp, thickening the hair, treating dandruff, smoothing the skin, treating seborrhea dermatitis, treating psoriasis and treating like conditions, possibly including healing of wounds in the skin and like uses is provided. Such a wide variety of applications is accordingly contemplated by the present invention and is to be understood as being intended by references to methods and/or compositions for treating hair and skin conditions.

In an embodiment of the invention, the composition of the invention comprises a phyto-steroid, such as, for example without limitation, is included in Glycyrrhiza Glaba-Papilionaceae. In another embodiment it the composition comprises Glycyrrhiza Glaba and an extract from at least one another herb.

To meet the objectives of the patent and the various types and causes of hair loss, the combination of herbs is base on the following:

GLYCYRRHIZA GLABA (Papilionaceae)—corticosteroid chemical structure, stimulate the adrenal cortex (Fahmy 1956), with estrogen like characters (Moaton 1978), anti-inflammatory and stress-relief. Active compounds are: Glycyrrhizin and Glycerretic acid.

LAVANDULA ANGUSTIFOLIA (Lavender)—stress-relief, stimulates the blood circulation to the follicles and strengthens them. Contains etheric oils such as: Lianolin acetate, Linalol, Cornalol, Camphor, Limonene.

FOENILULUM OFFICINALE (Umbelliferae)—stimulates the blood circulation to the follicles and strengthens them, anti-inflammatory. Active compounds are: etheric oils, fatty acids, flavanoides, vitamins and minerals.

SALVIA OFFICINALIS (Labiatae)—antiseptic, relief of the peripheral blood vessels and antibiotic activity. Active compounds are: etheric oils, flavanoides, Phenolic acids, estrogen like compounds.

ZINGIBER OFFICINALIS (Zingiberaceae)—stimulates the blood circulation, relief of the peripheral blood vessels and antiseptic. Active compounds are: etheric oils, Phenoles, alkaloids.

PASSIFLORA INCARNATA—sedative, given as non-addictive drug for restlessness, sleep disruptions, nervousness and mussels wrinkle in spastic disorder. Active compounds are: alkaloids, flavanoids, maltolistrols

It is a further object of the invention to provide methods and products for treating hair and skin conditions, wherein a treatment composition is topically applied to the skin and/or hair and hair follicles, the treatment agent comprises tinctures or extracts of one or more herbs selected from the class Papilionaceae, and optionally comprising at least one herb selected from any of the following classes: Lavender, Umbelliferae, Labiatae, Zingiberaleae, Passiflora and mixtures thereof.

It is a further object of the invention that the methods and products provided for treating hair and skin conditions, the treatment agent including tinctures or extracts of one or more herbs selected from the class Papilionaceae which may be Glycyrrhiza Glaba, and optionally comprising at least one of the following: Lavandula Avandula Angustifolia (Lavender), Foeniluium Officinale (Umbelliferae), Salvia Officinalis (Labiatae), Zingiber Officinalis (Zingiberaleae), Passiflora Incarnata and mixtures thereof.

In an embodiment of the invention, the invention contemplates mixtures of at least two or more of the above class of ingredients, in combination with Glycyrrhiza Glaba in the treatment composition. Such ingredients have been found to be particularly effective for synergistically enhancing treatment effectiveness and to facilitate penetration of the treatment composition into the skin, hair or hair follicles.

Although the present invention preferably contemplates topical application to the hair and/or skin to be treated, it is noted that such an application is generally believed necessary in order to provide an adequate concentration of the necessary ingredients in the local area of hair and/or skin to be treated. However, it is assumed that hair and/or skin treatment by such topical application may be enhanced or supplemented by additional internal consumption of the same herbs.

The herbs discussed above, particularly Glycyrrhiza Glaba, are preferably included in combination either with each other or with other herbs selected for synergistically enhancing treatment and/or delivery through the skin or hair follicles.

It is yet a further object of the invention to provide methods and products for treating hair and skin conditions including any of the combinations of ingredients and/or herbs noted above wherein the treatment composition further includes optional amounts of a carrier, a masking agent, a gelling agent, fragrances and preservatives as desired and a tincture solvent for the treatment agent.

Of the optional components noted above, the carrier provides a medium for the treatment agent and other components of the treatment composition as well as possible masking a strong smell possible for certain of the treatment agents.

In one embodiment, the invention provides a method for treating alopecia, promoting hair growth, preventing, stopping or minimizing hair loss, conditioning the hair, thickening the hair, comprising the step of administering an effective amount of a composition which comprises at least one phyto-corticosteroid and comprising at least one herb from the list mentioned above and at least one another acceptable pharmaceutical excipient.

In another embodiment the invention provides a method for treating dandruff, seborrhea dermatitis or psoriasis, comprising the step of administering an effective amount of a composition which comprises at least one phyto-corticosteroid and comprising at least one herb from the list mentioned above and at least one another acceptable pharmaceutical excipient.

In one embodiment of the invention, the term alopecia refers hereto to alopecia areata, alopecia totalis, alopecia universalis, male pattern alopecia, scaring alopecia, stress related alopecia and alopecia related to non-sufficient blood supply to the follicles and seborrhea.

In another embodiment of the invention, the phyto-corticosteroid may be derived from Glycyrrhiza Glaba extract (tincture).

In another embodiment of the invention, the phyto-corticosteroid may be derived from Glycyrrhiza Glaba extract (tincture) in alcohol and the phyto-corticosteroid amount is from 0.3% to about 100% of Glycyrrhiza Glaba.

In another embodiment of the invention, the alcohol may be but not limited to alcohol 70% (v/v).

In another embodiment of the invention, the alcohol may be but not limited to alcohol 95% (v/v).

In another embodiment of the invention, the alcohol may be but not limited to alcohol >95% (v/v).

In another embodiment of the invention, the herbal ingredients may be derived from Zingiber Officinalis extract (tincture).

In another embodiment of the invention, the herbal ingredients may be derived from Zingiber Officinalis extract (tincture) in alcohol and the herbal ingredients amount are from 0.1% to about 50% of Zingiber Officinalis.

In another embodiment of the invention the herbal ingredients may be derived from Zingiber Officinalis extract (tincture) in alcohol and the herbal ingredients amount are from 0.5% to about 20% of Zingiber Officinalis.

In another embodiment of the invention the herbal ingredients may be derived from Foeniculum Officinalis extract (tincture).

In another embodiment of the invention the herbal ingredients may be derived from Foeniculum Officinalis extract (tincture) in alcohol and the herbal ingredients amount is from 0.1% to about 20% of Foeniculum Officinalis.

In another embodiment of the invention the herbal ingredients may be derived from Foeniculum Officinalis extract (tincture) in alcohol and the herbal ingredients amount is from 0.2% to about 5% of Foeniculum Officinalis.

In another embodiment of the invention the herbal ingredients may be derived from Salvia Officinalis extract (tincture).

In another embodiment of the invention the herbal ingredients may be derived from Salvia Officinalis extract (tincture) in alcohol and the herbal ingredients amount is from 0.2% to about 50% of Salvia Officinalis.

In another embodiment of the invention the herbal ingredients may be derived from Salvia Officinalis extract (tincture) in alcohol and the herbal ingredients amount is from 0.5% to about 20% of Salvia Officinalis.

In another embodiment of the invention the herbal ingredients may be derived from Lavandula Angustifolia extract (tincture).

In another embodiment of the invention the herbal ingredients may be derived from Lavandula Angustifolia extract (tincture) in alcohol and the herbal ingredients amount is from 0.2% to about 30% of Lavandula Angustifolia.

In another embodiment of the invention the herbal ingredients may be derived from Lavandula Angustifolia extract (tincture) in alcohol and the herbal ingredients amount is from 0.4% to about 15% of Lavandula Angustifolia.

In another embodiment of the invention the herbal ingredients may be derived from Passiflora Incarnata extract (tincture).

In another embodiment of the invention there is provided a method, wherein the herbal ingredients may be derived from Passiflora Incarnata extract (tincture) in alcohol and the herbal ingredients amount are from 0.2% to about 30% of Passiflora Incarnata.

In another embodiment of the invention there is provided a method, wherein the herbal ingredients may be derived from Passiflora Incarnata extract (tincture) in alcohol and the herbal ingredients amount are from 0.5% to about 15% of Passiflora Incarnata.

In another embodiment of the invention the phyto-corticosteroid may be derived from Glycyrrhiza Glaba extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention there is provided a method wherein the herbal ingredients may be derived from Zingiber Officinalis extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention the herbal ingredients may be derived from Foeniculum Officinalis extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention the herbal ingredients may be derived from Saivia Officinalis extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention the herbal ingredients may be derived from Lavandula Angustifolia extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention the herbal ingredients may be derived from Passiflora Incarnata extract, which comprises isopropyl myristate (IPM): 0.2-3%, alcohol: 3-20%, biotine: 0.01-0.02%, vitamin E: 0.01-0.02%, mastic gum: 0.03-0.6%.

In another embodiment of the invention the composition may comprise phyto-corticosteroid that may be derived from Glycyrrhiza Glaba extract, gel components including (preservative, carbomer, trietanol amine, methyl dibromo, glutaronitrile, phenoxy ethanol), and sulfur, amino acids and purified water to balance up to 100% by weight

In another embodiment of the invention the herbal ingredients may be derived from Zingiber Officinalis extract, which comprises all components of gel including (preservative, carbomer, trietanol amine, methyl dibromo, glutaronitrile, phenoxy ethanol), and sulfur, amino acids and purified water to balance up to 100% by weight.

In another embodiment of the invention the herbal ingredients may be derived from Foeniculum Officinalis extract, which comprises all components of gel including (preservative, carbomer, trietanol amine, methyl dibromo, glutaronitrile, phenoxy ethanol), and sulfur, amino acids and purified water to balance up to 100% by weight.

In another embodiment of the invention the herbal ingredients may be derived from Salvia Officinalis extract, which comprises all components of gel including (preservative, carbomer, trietanol amine, methyl dibromo, glutaronitrile, phenoxy ethanol), and sulfur, amino acids and purified water to balance up to 100% by weight.

In another embodiment of the invention the herbal ingredients may be derived from Lavandula Angustifolia extract, which comprises all components of gel including (preservative, carbomer, trietanol amine, methyl dibromo, glutaronitrile, phenoxy ethanol), and sulfur, amino acids and purified water to balance up to 100% by weight.

In another embodiment of the invention the herbal ingredients may be derived from Passiflora Incarnata extract, which comprises all components of gel Including (preservative, carbomer, trietanol amine, methyl dibromo, glutaronitrile, phenoxy ethanol), and sulfur, amino acids and purified water to balance up to 100% by weight.

### DESCRIPTION OF DETAILED EMBODIMENTS

Methods and compositions for treating hair and skin conditions as summarized above are discussed in detail below.

In one embodiment, the invention provides a method for treating alopecia, promoting hair growth, preventing, stopping or minimizing hair loss, conditioning the hair, thickening the hair, comprising the step of administering an effective amount of a composition which comprises at least one phyto-corticosteroid and at least one other acceptable pharmaceutical excipient.

Although the invention contemplates in one of its embodiment to topical application to the hair and/or skin to be treated, it is noted that such an application is generally believed necessary in order to provide an adequate concentration of the necessary ingredients in the local area of hair and/or skin to be treated. However, it is assumed that hair and/or skin treatment by such topical application may be enhanced or supplemented by additional internal consumption of the same herbs.

In one embodiment, the invention contemplates mixtures of Glycyrrhiza Glaba and at least one or more of the above class of herbal ingredients, in combination with each other, in the composition of the invention. Such ingredients have been found to be particularly effective for enhancing treatment effectiveness and to facilitate penetration of the treatment composition into the skin, hair or hair follicles.

In another embodiment of the invention, the above herbs are employed in the treatment composition in various combinations with each other and/or with other herbs selected for enhancing treatment and/or delivery through the skin, hair or hair follicles. For example in the case of alopecia areata the combination of the phyto corticosteroid from Glycyrrhiza Glaba and the sedative effect of Passiflora Incarnata enhance the treating effect.

It is yet a further object of the invention to provide methods and products for treating hair and skin conditions including any of the combinations of herbal ingredients and/or herbs noted above wherein the treatment composition further includes optional amounts of a carrier, a masking agent, a gelling agent, fragrances and preservatives as desired and a tincture solvent for the treatment agent.

The gelling agent is of a type commonly employed in cosmetics and medicinal compositions for controlling the consistency or other physical characteristics of the treatment composition. The tincture solvent or a combination of different tincture solvents may be employed for forming the tinctures or extracts of the herbal ingredients and/or herbs comprising the treatment agent. A number of solvents including alcohol, IPM (Isopropyl myristate), purified water, etc. may be used for preparing the tinctures.

In any event, the methods and products of the present invention have been found to be surprisingly effective in the treatment of hair and skin conditions as noted above and discussed in greater detail below.

Additional objects and advantages of the invention are made apparent in the following description and examples of the invention.

The beneficial effects of the methods of the invention are demonstrated in the Examples section below.

In an embodiment of the invention the composition comprises Glycyrrhiza Glaba extract (tincture) in alcohol: 0.3—nearly 100% and at least one of the following agents according to the amounts described below or any combination thereof:
Zingiber Officinalis extract (tincture) in alcohol: 0.5-20%
Foeniculum Officinalis extract (tincture) in alcohol: 0.2-5%
Salvia Officinalis extract (tincture) in alcohol: 0.5-20%
Lavandula Angustifolia extract (tincture) in alcohol: 0.4-15%
Passiflora Incarnata extract (tincture) in alcohol: 0.5-15%

In an embodiment of the invention, the composition further comprises:
Isopropyl myristate (IPM): 0.2-3%
Alcohol: 3-20%
Biotine: 0.01-0.02%
Vitamin E: 0.01-0.02%
Mastic gum: 0.03-0.6%

Table 1 represents amounts of the chemicals discussed above in a variety of different herbs. Thus, in accordance with the preceding description, Table 1 indicates a possible order of preference for certain herbs.

TABLE 1

| | | | | | Enhancing | |
|---|---|---|---|---|---|---|
| | | Phyto- | Antiseptic | Relaxation | blood- | Other |
| | Cortico steroids | estrogens | features | features | circulation | features |
| Glycyrrhiza | + | + | + | + | | |
| Glaba | | | | | | |
| Lavandula | | | | + | + | |
| Angustifolia | | | | | | |
| Salvia | | + | + | | + | |
| Officinalis | | | | | | |
| Foeniculum | | + | + | | + | |
| Officinalis | | | | | | |
| Zingiber | | | + | | + | |
| Officinalis | | | | | | |
| Passiflora | | | | + | | |
| Incarnata | | | | | | |
| Water | | | | | | + |
| Carbomer | | | | | | + |
| Isopropyl | | | | | | + |
| myristate (IPM) | | | | | | |
| Trietanol | | | | | | + |
| amine | | | | | | |
| Alcohol | | | + | | | + |
| Sulfur | | | + | | | + |
| Methyl | | | | | | + |
| dibromo | | | | | | |
| Glutaronitrile | | | | | | + |
| Phenoxy ethanol | | | | | | + |
| L-lysine | | | | | | + |
| L-cysteine | | | | | | + |
| L-methionine | | | | | | + |
| L-Arginine | | | | | | + |
| Biotine | | | | | | + |
| Vitamin E | | | | | | + |
| Mastic gum | | | | | | + |

The methods and products of the present invention contemplate topical application of the treatment product to the skin or scalp, hair and hair follicles to be treated. Typically, about 10-100 cubic centimeters (cc) of the treatment product, was applied to the skin or scalp in test treatments.

However, smaller and larger amounts are also effective.

Furthermore, the treatment agent made up only a small portion of the treatment product during these tests. Generally, it is contemplated that the treatment agent in tincture form, may comprise as little as 3% or even as low as 1% by weight of the treatment product. More typically, the invention contemplates that the treatment agent in tincture form comprises in one embodiment in the range of about 1%-50% by weight of the treatment composition, in another embodiment of the invention about 3%-30% by weight of the treatment composition, and in another embodiment of the invention about 5%-15% by weight. However, the above concentrations are set forth only to define a preferred treatment product. It is again noted that a treatment product according to the present invention could include a very wide concentration range of the treatment agent in tincture form, depending upon concentration of the tincture and the specific therapeutic goal desired.

It is emphasized that the invention contemplates a very broad spectrum, as noted above, depending upon the concentration of the tincture in the final product. In one embodiment, for example, the tincture or extract may preferably range from a ratio of 50 parts of solvent to 1 part of ground herbs extract to a ratio of 1 part of solvent to 2 parts of ground herbs extract. The solvent is of course not limited to Alcohol but may be other suitable solvents as well, as discussed elsewhere herein.

The concentration of the tincture in the final product may vary, in one embodiment, is, for example, from 0.5 percent or as low as even 0.2 percent by weight, when using a more concentrated tincture, to almost 100 percent, in another embodiment of the invention, when using a more dilute tincture within the range mentioned.

Thus, the effective amounts of the active ingredients in the treatment agent are of course much smaller than the amount of the extract in the final product since only a small portion of the treatment agent in tincture form actually consists of the active ingredient. It is again noted that the active ingredients are selected from the class of herbal ingredients may be consisting of glycyrrhizin, glycerretic acid, analogs of the above and mixtures thereof.

In summary, the effective amount of the active ingredients in the final product equals the amount of the active ingredients in the tincture/extract (in the preferred broad range specified for solvent: treatment agent (Glycyrrhiza Glaba) equaling 50 to 1:2) multiplied by the percentage of the tincture in the final product (0.2% to 100% as noted elsewhere). It is further noted that, if a chemical or chemicals forming the active ingredients of the treatment agent are synthesized or isolated and not part of a natural herbal extract, then the range of amounts and concentrations can be even broader than discussed above.

It is specifically to be noted that the present invention essentially requires topical (or external) application of the composition to the skin or hair of a host in need thereof. Such external treatment is contemplated for use by itself. In fact, tests conducted for the invention included only such external treatment. However, internal treatment may be used but only as a supplement to external or topical treatment as defined herein. Specifically, supplemental internal treatment is optional and not necessary to the invention while internal treatment by itself is not contemplated for the invention.

As for frequency of application, it is typically contemplated that the treatment product, in the form of either a hair cream or skin cream, or liquid or other form, for example, be applied from about twice a day to about twice a month to the skin area or scalp area being treated, and taken internally as well to possibly supplement or enhance external treatment. However, effective treatment is also possible with a frequency of application of, for example, from a maximum of three to four times daily or even more frequently to once a month or even less frequently.

Effective topical treatment appears to be from about once daily to once every seven days with the applied composition remaining in place for at least about 15-30 minutes and preferably for a period of about 8-10 hours or more.

Particularly in terms of the method of treatment for the invention, it is noted that most tests conducted were in connection with promoting hair growth or preventing, stopping or minimizing hair loss. With the methods and products of the invention being employed for other purposed, particularly for treating various skin conditions, it is possible that different application amounts and rates may be desirable. In particular, it may be found that substantially different amounts, probably smaller quantities, may be desirable for treating certain skin conditions; however, it is again emphasized that the optimum amounts and frequencies of application are not specifically known for all applications.

### EXAMPLES

### Example I

### Preparation of the Composition

Some of the experiments shown below were carried out with a treatment product including a treatment agent formed as a tincture of Glycyrrhiza Glaba in combination with a tincture of Zingiber Officinalis, Foeniculum Officinalis, Salvia Officinalis, Lavandula Angustifolia, Passiflora Incarnata or combination thereof.

The primary herb, Glycyrrhiza Glaba, was first grounded into a powder or cut into pieces and soaked into organic solvent (alcohol or IPM—Isopropyl myristate), an organic solvent in a ratio of about one part of Glycyrrhiza Glaba to three parts of alcohol solvent.

Vegetable gum, or gum mastic, was dispersed separately into Isopropyl myristate solvent in a ratio of about ten parts IPM solvent and one part gum mastic.

The Glycyrrhiza Glaba tincture wasallowed to stand for about two weeks, mixing the tincture from time to time. After two weeks, the tincture was filtered to produce a clear liquid.

The clear Glycyrrhiza Glaba tincture was mixed in a blender with the dispersed mastic gum in a ratio of about twenty parts Glycyrrhiza Glaba tincture and about one part gum mastic solution to produce a Glycyrrhiza Glaba tincture solution.

A second tincture was prepared by extracting the herbs Lavandula Angustifolia (Lavender), Foenilulum Officinale, Salvia Officinalis (Labiatae), Zingiber Officinalis (Zingiberaleae) and Passiflora Incarnata with alcohol as solvent. These herbs were in ratio 1:1:1:1:1 and the ratio between them and the solvent 1 (herbs): 15 (solvent). This tincture was also allowed to stand for two weeks, with stirring. Thereafter, the tincture was filtered to also produce a clear liquid.

The two extracts (Glycyrrhiza Glaba) and all the other herbs Lavandula Angustifolia (Lavender), Foenilulum Officinale, Salvia Officinalis (Labiatae), Zingiber Officinalis (Zingiberaleae) and Passiflora Incarnata were mixed in a blender in ratio 1:1 and then this mixture (20%) was mixed in a blender with all the other ingredients to form a combined mixture (80%).

The above components combined as described formed the basic active product for the treatment product of the invention. The basic active product was mixed with a gel in a ratio of about one part of basic active product and about twelve parts of gel. Any of a number of well known gels may be used with the invention.

In any event, the gelling agent is added as the last component of the composition in order to produce the preferred gel form for the composition.

Thus the final concentration includes:
Glycyrrhiza Glaba extract (tincture) in alcohol: 0.3—nearly 100%
Zingiber Officinalis extract (tincture) in alcohol: 0.5-20%
Foeniculum Officinalis extract (Uncture) in alcohol: 0.2-5%
Salvia Officinalis extract (tincture) in alcohol: 0.5-20%
Lavandula Angustifolia extract (tincture) in alcohol: 0.4-15%
Passiflora Incarnata extract (tincture) in alcohol: 0.5-15%
Isopropyl myristate (IPM): 0.2-3%
Alcohol: 3-12%
Biotine: 0.01-0.02%
Vitamin E: 0.01-0.02%
Mastic gum: 0.03-0.6%
All components of gel including (preservative, carbomer, trietanol amine, methyl dibromo, glutaronitrile, phenoxy ethanol), and sulfur, amino acids and purified water to balance up to 100% by weight.

### Example II

### Test Results

A group consisting of more than seventy females and males suffering from various stages of baldness, hair and scalp problems was selected for the experiment. The patients received the treatment product described in Example I.

The following results were demonstrated:

Hair loss was halted within 2-6 weeks. Nearly 95% of the participants (of 74 subjects), both male and female, who complained of prior hair loss reported that the hair loss stopped.

After four months of treatment, 86% of the subjects (in total 73 subjects), reported of initiation of hair growth. Even in those few who did not report hair growth but who continued the treatment, initiation of hair growth was observed after about six months. After one year of treatment in both males and females, in generally all states of baldness, new hair growth was observed in over 85% of the subjects. These results were based upon tests with a group of 76 men and women.

The participants also commonly reported a substantial decrease in dandruff and stoppages of itch scalp 86% (of 22 subjects).

Reports of hair growth & thickening in about 84% of the participants (55 subjects) who complained of previous hair loss.

Female participants (47 subjects), new hair growth was observed in over 87% of the subjects, while male participants (26 subjects), new hair growth was observed in over 85% of the subjects.

Female participants (16 subjects) stopped of dandruff & itch scalp was observed in over 81% of the subjects, while male participants (6 subjects) all concords.

Female participants (40 subjects) initiation of hair growth & thickening was observed in over 83% of the subjects, while male participants (15 subjects), initiation of hair growth & thickening was observed in over 87% of the subjects.

It was generally noted almost all participants in tests according to the present invention reported improvement in hair quality, thickness and health.

The participants with seborrhea dermatitis (6 subjects) previously treated by other methods and compositions, reported of a substantial decrease in dandruff. Tests for psoriatic lesions in a few patients having those conditions also showed significant improvement. Excellent therapeutic results were identified by patients treated with the composition of the invention in all scalp and skin ailments including but not limited to itching, dandruff, seborrhea dermatitis and psoriasis of the scalp.

Continued testing according to Example I and other examples of the present invention have indicated that use of the present invention, particularly in males over a period of about six months has achieved nearly a 100% response in reported hair growth and termination of hair loss.

### Example III

### Further Test Procedures

More specific test procedures were carried out with twenty eight individuals, both male and female, from about 7 to about 70 years old. These subjects employed the treatment product prepared in accordance with Example I for periods of time ranging from six days to approximately six months. These subjects previously administered with different treatment (such as: Dermovat, Pluvitor, Dithrocream, cortisone injections or Minoxidil for Alopecia Totalis; cortisone injections, steroids, Dermovat, Beta Corten, Regain or Dithrocream for Alopecia Areata; Beta Corten, Diprosalic Acid, Regain, Alphosyl, Nizoral or Daivonex for Seborrhea; Alphosyl, Nizoral or Daivonex for Psoriasis; Nizoral or Agispor for Dandruff; Regain, Propecia, Nizoral, Androcur or Minoxidil for hair loss; or even no treatment at all) before starting with the treatment product as shown in Example 1. The treatment known products specified above, were without any or significant effect on hair loss or dandruff and itching scalp conditions. The subjects, at initiation of testing, had a variety of hair conditions ranging from substantially bald through receding hairlines to thinning hair and full heads of hair with varying degrees of hair falling out. Of 28 subjects employing the treatment product of the invention for more than two months, all reported on hair loss stoppage while 94% (of 26 subjects) reported new hair growth and all of them also reported hair growth & thickening at the same time. Further, 63% reported improve in condition of dandruff & itch scalp. These subjects experienced varying degrees of success ranging from substantial new hair growth to hair loss being .controlled at a normal rate for individuals with generally healthy heads of hair. In any event, the results of Examples II and III are believed to clearly demonstrate the efficacy and effectiveness of the treatment product of Example I.

### Example IV

### Treatment of Alopecia Totalis

More specific test procedures were carried out with five individuals, both male and female, ages 9, 12, 29, 39 and 69 years old. These subjects employed a composition with Glycyrrhiza Glaba and Zingiber Officinalis as active ingredients to stimulate the hair follicles and stimulate blood circulation, for periods of time ranging from six days to six months. These subjects suffer from alopecia totalis and were previously administered with a variety of different treatments such as Dermovat, Pluvitor, Dithrocream, cortisone injections or Minoxidil, which were not successful before starting with the treatment product of the present invention. These subjects, at initiation of testing, had a variety of hair conditions. All subjects reported of improvement in their hair condition, new hair growth and thickening of hair.

### Example V

### Treatment of Alopecia and Seborrhea or Seborrhea Dermatitis

More specific test procedures were carried out with five individuals, all female, ages 7, 33, 38, 40 and 57 years old. These subjects employed the treatment product as Example IV, including Glycyrrhiza Glaba and Zingiber Officinalis as active ingredients with the addition of sulfur, for periods of time ranging from as little as six days to as long as approximately six months. These subjects suffer from alopecia and seborrhea and were previously administered with a variety of different treatments such as Beta Corten, Diprosalic Acid, Regain, Alphosyl, Nizoral or Daivonex, which were not efficient before starting with the treatment product of the present invention. These subjects, at initiation of testing, had a variety of stages of alopecia and seborrhea. All subjects reported of improvement in hair condition, stoppage of hair lost, vanished of seborrhea, clean scalp and stop of itchiness.

### Example VI

### Treatment of Dandruff

More specific test procedures were carried out with two individuals, both female, aged 28 and 34 years old. These subjects employed the treatment product as in Example V, for periods of time ranging six days to approximately six months. These subjects suffer from dandruff and were previously administered with a variety of different treatments such as Nizoral or Agispor, which were not effective before starting with the treatment product of the present invention. The subjects, at initiation of testing, had variety stages of scalp condition. All subjects reported of improvement in hair condition, clean scalp, stoppage of itchiness and elimination of dandruff.

### Example VII

### Treatment of Alopecia Areata

More specific test procedures were carried out with six individuals, both male and female, aged 8, 12, 24, 32, 35 and 42 years old. These subjects employed the treatment product prepared in accordance with Example I, including Glycyrrhiza Glaba, Zingiber Officinalis, Foeniculum Officinalis, Salvia Officinalis, Lavandula Angustifolia, Passiflora Incarnata and amino-acids, for periods of time ranging from as little as six days to as long as approximately six months. These subjects suffer from alopecia areata and were previously administered with a variety of different treatments such as Cortisone injections, steroids, Dermovat, Beta Corten, Regain or Dithrocream, which were not effective before starting with the treatment product of the present invention. The subjects, at initiation of testing, had a variety of hair conditions. All subjects reported of improvement in hair condition, stoppage of hair lost, new hair growth and thickening of hair.

### Example VIII

### Treatment of Psoriasis

More specific test procedures were carried out with two individuals, both female, aged 52 and 57 years old. These subjects employed the treatment product prepared in accordance with Example V (similar ingredients as in seborrhea), for periods of time ranging from as little as six days to as long as approximately six months. These subjects suffered from psoriasis and were previously administered with a variety of different treatments such as Alphosyl, Nizoral or Daivonex, which were not effective before starting with the treatment product of the present invention. The subjects, at initiation of testing, had a variety of hair conditions. All subjects reported of improvement in hair condition, elimination or reduce of dandruff and total recovery of the Psoriasis.

What is claimed is:

1. A method for treating alopecia, promoting hair growth, preventing, stopping or minimizing hair loss, conditioning the hair, thickening the hair, comprising the step of administering an effective amount of a composition comprising at least one phyto-corticosteroid and at least one acceptable pharmaceutical excipient.

2. The method according to claim 1, wherein the alopecia is alopecia areata, alopecia totalis, alopecia universalis, male pattern alopecia, scaring alopecia, stress related alopecia and alopecia related to non-sufficient blood supply to the follicles.

3. The method according to claim 2, wherein the composition further comprises at least one herbal ingredient.

4. The method according to claim 1, wherein the phyto-corticosteroid is in an amount of 0.3% to about 100% and is derived from an extract comprising alcohol.

5. A method for treating dandruff, seborrhea dermatitis or psoriasis, comprising the step of administering an effective amount of a composition comprising at least one phyto-corticosteroid and at least one acceptable pharmaceutical excipient.

6. The method according to claim 5, wherein the composition further comprises at least one herbal ingredient.

7. The method according to claim 5, wherein the phyto-corticosteroid is in an amount of 0.3% to about 100% and is derived from an extract comprising alcohol.

8. The method according to claim 1, wherein the phyto-corticosteroid is derived form the class Papilionaceae.

9. The method according to claim 8, wherein said phyto-corticosteroid is derived from Glycyrrhiza Glaba extract (tincture).

10. The method according to claim 5, wherein the phyto-corticosteroid is derived form the class Papilionaceae.

11. The method according to claim 10, wherein said phyto-corticosteroid is derived from Glycyrrhiza Glaba extract (tincture).

12. The method according to claim 3, wherein the herbal ingredient is selected from the following classes: Lavender, Umbelliferae, Labiatae, Zingiberaleae, Passiflora and any combination thereof.

13. The method according to claim 12, wherein said herbal ingredient is derived from Zingiber Officinalis, Foeniculum Officinalis, Salvia Officinalis, Lavandula Angustifolia, or Passiflora Incarnata extract (tincture) or any combination thereof.

14. The method according to claim 6, wherein the herbal ingredient is selected from the following classes: Lavender, Umbelliferae, Labiatae, Zingiberaleae, Passiflora and any combination thereof.

15. The method according to claim 14, wherein said herbal ingredient is derived from Zingiber Officinalis, Foeniculum Officinalis, Salvia Officinalis, Lavandula Angustifolia, or Passiflora Incarnata extract (tincture) or any combination thereof.

16. The method according to claim 1, wherein the composition comprises isopropyl myristate (IPM): 0.2-3%, alcohol: 3-12%, biotine: 0.01-0.02%, vitamin E: 0.01-0.02%, mastic gum: 0.03-0.6%.

17. The method according to claim 5, wherein the composition comprises isopropyl myristate (IPM): 0.2-3%, alcohol: 3-12%, biotine: 0.01-0.02%, vitamin E: 0.01-0.02%, mastic gum: 0.03-0.6%.

18. The method according to claim 1, wherein the composition comprises preservative, carbomer, trietanol amine, methyl dibromo, glutaronitrile, phenoxy ethanol, sulfur, amino acids and purified water to balance up to 100% by weight.

19. The method according to claim 5, wherein the composition comprises preservative, carbomer, trietanol amine, methyl dibromo, glutaronitrile, phenoxy ethanol, sulfur, amino acids and purified water to balance up to 100% by weight.
